(19) United States
(12) Patent Application Publication
(10) Pub. No.: US 2021/0121205 A1
(43) Pub. Date: Apr. 29, 2021

(54) BONE IMPLANT, IN PARTICULAR FOR VERTEBRAL ARTHRODESIS, PROVIDED WITH BLOCKING MEANS FOR ANCHOR SCREW

(71) Applicant: ORTHOPAEDIC & SPINE DEVELOPMENT (OSD), Avignon (FR)

(72) Inventors: Stéphane RAMARE, Le Mans (FR); Patrick TROPIANO, Marseille (FR); Paolo MANGIONE, Bordeaux (FR); Olivier GILLE, Pessac (FR)

(73) Assignee: ORTHOPAEDIC & SPINE DEVELOPMENT (OSD), Avignon (FR)

(21) Appl. No.: 17/083,053

(22) Filed: Oct. 28, 2020

(30) Foreign Application Priority Data
Oct. 28, 2019 (FR) 19/12047

(51) Int. Cl. A61B 17/70; A61F 2/44

(52) U.S. Cl. CPC A61B 17/7059; A61F 2/4455; A61B 17/7076

(57) ABSTRACT

A bone implant including a plate with an orifice extending along a steering axis capable of receiving an anchor screw extending along a screw axis, where a first blocking member is arranged in a first peripheral housing formed in the orifice, and a second blocking member is arranged in a second peripheral housing formed in the orifice and offset relative to the first housing according to the steering axis, and the orifice includes a lower portion providing a bearing with polyaxiality to the anchor screw so that the screw axis can form with the steering axis a screw angle with an adjustable measurement, and the blocking members are elastically deformable between an open configuration, in which they authorize an insertion of the anchor screw, and a closed configuration, in which they prevent an extraction of the anchor screw regardless of the measurement of the screw angle.

The present invention relates to a bone implant provided to be fixed to one or more bone structures by means of anchor screws.

It also concerns anchor screws suitable for fixing such a bone implant to one or more bone structures.

In the surgical field, it is common to use bone implants in order to hold in place and stabilize bone structures, these bone implants generally being in the form of a plate including one or more orifices adapted to receive each an anchor screw for fixing said plate on said bone structures.

For example, such a bone implant can allow a stabilization of the spine by being fixed to two successive vertebrae, the bone implant thus forming a vertebral arthrodesis implant providing a fusion of two vertebrae.

It is also necessary to provide, in these bone implants, blocking means suitable for blocking the anchor screws in the orifices formed in the plate.

Indeed, once the anchor screws have been introduced into the bone structure(s), it is possible that they undergo a (partial or total) extraction outside the orifices of the bone implant, for example following significant mechanical stresses exerted in the bone structure(s), a deterioration in the density or quality of the bone structure(s), or even an imperfect implantation by the practitioner: this phenomenon of extracting the anchor screws, also called «backing-out», can cause a degradation in the quality of the fixation of the bone implant on the bone structure(s) (and therefore a degradation of the stabilization allowed by it) and cause damage to the tissues or surrounding organs.

These blocking means must also be able to authorize an insertion of the anchor screws into the orifices of the bone implant.

These blocking means may thus include, in a known manner, an elastic blocking member of the split ring type, disposed in a peripheral housing formed in an orifice of the bone implant and adapted to be in contact with an anchor screw received in this orifice, so as to prevent its extraction.

For example, the document WO2009/132305 describes a bone implant including a split ring disposed in a peripheral housing formed in an orifice receiving an anchor screw. This peripheral housing and this split ring have an oval shape: by rotating the split ring in the peripheral housing, it is possible to deform the split ring between an open configuration, in which it authorize the insertion of the anchor screw, and a closed configuration, in which it prevents an extraction of this same anchor screw.

Similarly, document WO2007/136452 describes a bone implant including two orifices adapted to receive each an anchor screw, each having a peripheral housing in which a split ring is disposed in contact with a central wheel: by rotating this wheel, it is possible to deform the two split rings in their respective peripheral housings between an open configuration, in which they authorize an insertion of the anchor screws, and a closed configuration, in which they prevent extraction of these same anchor screws.

The document US2015/0245859 describes a bone implant including two orifices adapted to receive each an anchor screw. Each orifice has a peripheral housing communicating with one another so as to form a double housing in the general shape of a «W», in which is disposed an elastic member formed of two split rings linked to one another and also in the general shape of a «W»: by displacing this elastic member in translation in the double housing, it is possible to deform the two split rings forming the elastic member between an open configuration, in which they authorize an insertion of the anchor screws, and a closed configuration, in which they prevent an extraction of these same anchor screws.

However, these three bone implants have the drawback that the change in the configuration of the split rings (between an open configuration, in which they authorize an insertion of the anchor screws, and a closed configuration, in which they prevent an extraction of these same anchor screws) is not automatic and requires specific action by the practitioner: the procedure for fixing these bone implants is therefore more complex and can be very inconvenient when they are implanted in areas that are difficult to access.

Particularly, in the context of so-called «minimally invasive» operating techniques, limiting the operative approach to an access route of only a few centimeters in diameter, it may be very difficult for the practitioner to reach the bone implants in order to actuate their blocking means.

In addition, these implants themselves have a complex geometry and a large number of movable parts: they are therefore fragile and expensive to manufacture.

Finally, the document FR2810532 describes a bone implant including two orifices adapted to receive each an anchor screw and a split ring disposed between these two orifices. This split ring can cooperate, via two lateral slots, with the anchor screws received in said orifices: it can be deformed between a closed configuration, in which it authorizes an insertion of the anchor screws, and an open configuration in which it prevents an extraction of these same anchor screws.

The split ring can here be automatically deformed during the insertion of the anchor screws without requiring any action by the practitioner, but this bone implant has the drawback that, due to the small contact surface between the ring split and the anchor screws, the split ring is not able to ensure effective and safe blocking of the anchor screws, in particular when the implantation orientation of these anchor screws can vary by one operation to another.

In addition, none of the bone implants described by the aforementioned documents makes it possible to reliably ensure a blocking with polyaxiality of the anchor screws, that is to say a blocking of the anchor screws when these can be introduced in different screwing directions in the orifices.

Indeed, it may be useful for the practitioner to adjust the direction of insertion of the anchor screws into the orifices and to modify their inclination, in order to improve the anchoring of the bone implant on the bone structure(s).

The blocking means described by the aforementioned documents only including a single split ring per orifice, they can hardly be adapted to the different inclinations of the anchor screws and effectively block their extraction.

The invention proposes to resolve all or part of these drawbacks, by proposing a bone implant provided with blocking means making it possible to effectively prevent an extraction of the anchor screws from the orifices in which they are received, even when said bone implant authorizes an insertion of said anchor screws in said orifices with polyaxiality.

Another object of the invention is to propose a bone implant provided with blocking means allowing alternatively to authorize an insertion of the anchor screws in the orifices of said implant and to prevent an extraction of the latter, without requiring a specific action of the practitioner during the placement of said bone implant.

Yet another object of the invention is to propose a bone implant which is simple to manufacture and to use.

To this end, it proposes a bone implant, adapted to be fixed on at least one bone structure, including a plate provided with at least one anchor assembly provided with an orifice extending along a steering axis and capable of receiving an anchor screw extending along a screw axis, said anchor assembly comprising a first blocking member disposed in a first peripheral housing formed in said orifice,

said bone implant being characterized in that:
said anchor assembly further comprises at least one second blocking member disposed in a second peripheral housing formed in said orifice, said second peripheral housing being offset relative to the first peripheral housing along the steering axis,
said orifice includes a lower portion offering, when said anchor screw is received in said orifice, a bearing with polyaxiality to said anchor screw so that the screw axis can form a screw angle with the steering axis to the adjustable measure, and
said first blocking member and second blocking member are elastically deformable between an open configuration, in which they authorize insertion of said anchor screw into said orifice, and at least one closed configuration, in which they prevent extraction of said anchor screw out of said orifice, regardless of the measurement of the screw angle.

The orifice of such a bone implant is thus adapted to receive an anchor screw, allowing the bone implant to be fixed on a bone structure, and has an inner structure authorizing a polyaxiality of this anchor screw.

Indeed, the lower portion of the orifice has a bearing surface on which is intended to bear an abutment of the anchor screw, for example so as to form a linear contact between these two elements: by ensuring that the lower portion has a sufficient width to allow movement of the anchor screw inside the orifice of the bone implant, it is then possible to incline the anchor screw relative to the steering axis of the orifice, while maintaining contact between the anchor screw and the lower portion.

The anchor screw can thus extend along the steering axis and in at least one screwing direction which is oblique with respect to the steering axis, that is to say forming a screw angle with this same steering axis, of non-zero measurement: at least two inclination configurations of the anchor screw in the orifice are therefore possible.

The measurement of the screw angle can therefore be adjusted by the practitioner, during the operation of placing the bone implant, in a cone of revolution centered on the steering axis of the orifice: this possibility allows a greater freedom of action for the practitioner, who can adapt the screwing direction of the anchor screw, for example to the particular geometry of the bone structure on which the bone implant is fixed, or to the mechanical constraints exerted on said bone implant.

The particular structure of the implant according to the invention, by authorizing a polyaxiality of the anchor screw received in the orifice of the plate, thus makes it possible to facilitate the placement of the bone implant by the practitioner and to improve the quality of its fixation.

According to a characteristic, the lower portion of the orifice has a generally spherical or frustoconical shape.

Moreover, the bone implant according to the invention is designed to allow effective blocking of the anchor screw in the orifice, regardless of the inclination position of the anchor screw.

Indeed, it includes two blocking members, disposed in peripheral housings formed in a wall of the orifice, and positioned one above the other along the steering axis of the orifice.

These blocking members are moreover suitable for cooperating with the anchor screw received in the orifice by coming into physical contact with the latter: when the blocking members are in an open configuration, they authorize the insertion of the anchor screw in the orifice (and therefore the operation of screwing said anchor screw into the bone structure on which the bone implant is fixed), and when they are in a closed configuration, they prevent, by their contact with the anchor screw, an extraction of the same anchor screw from the orifice.

The anchor screw, once it has been inserted into the orifice, is therefore in contact with at least the first blocking member and the second blocking member (each being in a closed configuration), regardless of the inclination of this anchor screw with respect to the steering axis (that is to say, whatever the measurement of the screw angle): thanks to these at least two contact points offset according to the steering axis of the orifice, the anchor screw can be securely held in position in the orifice and an extraction movement thereof is prevented.

Thus, compared to the implants described by the aforementioned prior documents which only include a single blocking member, the bone implant according to the invention therefore makes it possible to improve the reliability of the blocking of the anchor screw in the orifice of the bone implant, particularly when the screw angle has a non-zero measurement.

It will be noted that it is conceivable that, depending on the geometry of the lower portion of the orifice and the interaction between the anchor screw and the first blocking member and the second blocking member, the screw angle can take any value between 0 degrees (the anchor screw then extends along the steering axis) and a maximum screw angle value, or else it can only take a limited number of values in this same interval.

According to a possibility, the screw angle measurement is adjustable within a range of 0 to 15 degrees.

Advantageously, the anchor assembly can have one or more additional blocking members, each disposed in a peripheral housing offset relative to the other peripheral housings along the steering axis of the orifice: by increasing the number of blocking members coming into contact with the anchor screw received in the orifice, it is possible to improve the quality of the blocking thereof in this same orifice.

For example, the bone implant may include a third blocking member disposed in a third peripheral housing offset with respect to the first peripheral housing and the second peripheral housing along the steering axis.

In an embodiment, the first blocking member and the second blocking member are elastically deformable in the sense of a natural return from the open configuration to a closed configuration in the absence of an external constraint applied to them.

In this way, the first blocking member and the second blocking member cannot remain spontaneously in the open configuration and constantly tend to be deformed towards a closed configuration: they are only kept in the open configuration under the effect of an external constraint.

Thus, the first blocking member and the second blocking member allow a spontaneous and automatic blocking of the anchor screw in the orifice of the bone implant because, in the absence of constraint exerted on them by the practitioner, they are deformed towards a closed configuration in which they are in contact with the anchor screw and prevent an extraction of the latter outside the orifice.

This characteristic allows to greatly facilitate the fixation of the bone implant on a bone structure: the practitioner just has to screw, by a usual gesture, the anchor screws in the orifice, and therefore, they naturally exert on the first blocking member and the second blocking member a constraint during the insertion of the anchor screw into the orifice (the first blocking member and the second blocking member are then in the open configuration and authorize such an insertion), this constraint on said first blocking member and second blocking member being released by itself once the anchor screw screwed into the bone structure, these coming to block said anchor screw in the orifice and prevent its extraction (the first blocking member and the second blocking member are then deformed towards a closed configuration, in which they prevent any extraction movement).

The practitioner therefore does not need, unlike the already known bone implants, to manually deform the blocking members to lock the anchor screw in the orifice, which can be an inconvenient operation to perform.

It should be noted that it is conceivable that the first blocking member and the second blocking member have a single open configuration, but a plurality of closed configurations: depending on the geometry of the anchor screw and its position (inclination) in the orifice, the first blocking member and the second blocking member will undergo more or less significant elastic deformations before coming into contact with this anchor screw and the blocking of the latter will be ensured by different closed configurations.

Alternatively, the first blocking member and the second blocking member are each of the split ring or circlip type.

This type of blocking member has the advantage of being very simple to make.

The anchor screw can then be received in the center of such a split ring or circlip: the change from a closed configuration to the open configuration results in an increase in an inner diameter of the split ring or the circlip up to a maximum internal diameter, where the split ring or the circlip loses physical contact with the anchor screw.

Conversely, the passage from the open configuration to a closed configuration then results in a reduction of an inner diameter of the split ring or the circlip, until the split ring or the circlip comes into contact with the anchor screw and can no longer be further deformed.

According to a characteristic, the first blocking member and the second blocking member are identical.

According to a possibility, the first blocking member and the second blocking member each extend in a direction orthogonal to the steering axis of the orifice.

It should be noted that the first blocking member and the second blocking member are then parallel to each other and the anchor screw has the same inclination with respect to each of them.

In an embodiment, each of the first blocking member and of the second blocking member has at least two distinct radial projections, said radial projections being intended to be brought into contact with the anchor screw when said anchor screw is received in the orifice and when said first blocking member and said second blocking member are in a closed configuration.

These radial projections constitute distinct contact surfaces, distributed around the anchor screw when the latter is received in the orifice, whose contact with this same anchor screw makes it possible to prevent extraction from the orifice.

The presence of several distinct contact surfaces allows the first blocking member and the second blocking member to be easily adapted to the geometries and to the particular orientations of the anchor screws which can be received in the orifice, while guaranteeing a number of sufficient contact points to effectively hold the anchor screw in the orifice.

Particularly, when the anchor screw includes projecting structures on its periphery, for example stop detents or stoppers, the radial projections of the first blocking member and of the second blocking member can cooperate in a simple manner with these projecting structures, without the first blocking member and the second blocking member having to match the shape of the anchor screw and cooperate with the latter over the entire periphery.

In addition, due to the possibility of polyaxiality for the same anchor screw in the orifice, each blocking member must be able to be adapted to a different geometry of the anchor screw depending on the screw angle formed by the latter with the steering axis: the presence of their respective radial projections gives the first blocking member and the second blocking member a good ability to be adapted to these different configurations, while ensuring permanent effective blocking of the anchor screw in the orifice.

According to a possibility, each of the first blocking member and of the second blocking member has at least four distinct radial projections.

According to a characteristic, the radial projections are distributed over the first blocking member and the second blocking member so that they are symmetrical with respect to an axial plane of symmetry, such as for example a plane of symmetry including the steering axis.

According to another characteristic, the radial projections of the first blocking member and the radial projections of the second blocking member are not aligned (or superimposed) in a direction parallel to the steering axis.

Advantageously, the plate includes at least two anchor assemblies.

Each of these anchor assemblies has, as previously described, a first blocking member and a second blocking member and is adapted to receive an anchor screw whose inclination relative to the steering axis of each orifice can be adjusted: it is thus possible to fix the bone implant on several distinct bone structures, the anchor assemblies authorizing an insertion with polyaxiality of each anchor screw then preventing an extraction of these same anchor screws out of the orifices in which they are respectively received.

For example, the bone implant can have two anchor assemblies and be securely attached to two distinct bone structures.

The invention also concerns an anchor screw suitable for a bone implant as previously described, said anchor screw being adapted to cooperate with the anchor assembly of said bone implant, said anchor screw having a rod extending along a screw axis and a head provided with:
a lower abutment suitable to bear with polyaxiality on the lower portion of the orifice of said anchor assembly so that, when said anchor screw is received in said orifice, the screw axis can form with the steering axis a screw angle with an adjustable measurement; and
at least two unidirectional detents, said unidirectional detents being provided to cooperate with the first blocking member and the second blocking member when said anchor screw is received in the orifice of said anchor assembly, so that said first blocking member and the second blocking member authorize an insertion of said anchor screw in said orifice when they are in the open configuration and prevent an extraction of said anchor screw out of said orifice when they are in a closed configuration, whatever the measurement of the screw angle.

Such an anchor screw is thus particularly suitable for being associated with a bone implant according to the invention, the unidirectional detents having a structure allowing, by their interaction with the first blocking member and the second blocking member when the anchor screw is received in the orifice of the bone implant, an insertion of the screw of said orifice and preventing an extraction of this same anchor screw out thereof.

Moreover, this anchor screw also has a geometry allowing it to be inserted with polyaxiality into the orifice of the bone implant: the screw head can be brought into contact with the lower portion of the orifice, thus authorizing a pivoting with respect to the steering axis of the orifice and adjustment of the screw angle.

According to a possibility, the lower surface of the anchor screw has a shape complementary to the lower portion of the orifice of the bone implant.

Advantageously, the unidirectional detents have a symmetry of revolution centered on the screw axis.

In this way, these unidirectional detents can cooperate with the first blocking member and the second blocking member regardless of the orientation of the anchor screw in the orifice of the implant.

For example, the lower abutment of the anchor screw may have a spherical or frustoconical shape.

In an embodiment, the unidirectional detents are formed by:
blocking plates, successively disposed around the head of said anchor screw, mutually parallel and orthogonal to the screw axis, and
side ramps joining two successive blocking plates,

said blocking plates allowing, by contact with the first blocking member and the second blocking member when said anchor screw is received in the orifice of the bone implant and when said first blocking member and said second blocking member are in a closed configuration, to prevent extraction of said anchor screw from said orifice of said bone implant.

The anchor screw thus has several successive unidirectional detents along the screw axis, each unidirectional detent being formed by a blocking plate and a lateral ramp.

Each unidirectional detent can thus appear as an indentation practiced in the anchor screw intended to receive the first blocking member or the second blocking member.

Particularly, the blocking plates extend orthogonally to the screw axis, and are therefore also orthogonal to the steering axis of the bone implant when the anchor screw is inserted into the orifice at a screw angle of a zero measurement.

These blocking plates are also substantially orthogonal to the steering axis when the anchor screw is inserted into the orifice at a low measurement screw angle, which corresponds to the majority of practical cases.

Thus, when these blocking plates are brought into contact with the first blocking member and the second blocking member, they effectively make it possible to prevent an extraction of the anchor screw from the orifice of the bone implant, by their orthogonality to the steering axis.

According to a possibility, the lateral ramps are configured to apply, during an insertion movement of said anchor screw in the orifice of the bone implant, a constraint on the first blocking member and the second blocking member, said constraint making it possible to deform said first blocking member and second blocking member so as to change them from a closed configuration to the open configuration.

For example, the lateral ramps can be in the form of frustoconical or hemispherical surfaces joining the successive blocking plates and presenting an oblique direction with respect to the screw axis: these lateral ramps therefore appear as a progressive widening of the diameter of the anchor screw, between each blocking plate, in the direction going from the rod towards the head of the anchor screw.

In this way, during the insertion of the anchor screw into the orifice, these lateral ramps have the effect of exerting a radial constraint on the first blocking member and the second blocking member in contact with the blocking screw: under the effect of this constraint, the latter are deformed and change from a closed configuration to the open configuration, thus allowing the movement of insertion of the anchor screw in the orifice.

Conversely, each blocking plate constituting a sudden reduction in the diameter of the anchor screw, in the direction from the rod towards the head of the anchor screw, the first blocking member and the second blocking member are abruptly deformed from the open configuration towards the closed configuration when they come into contact with a blocking plate at the end of a lateral ramp.

During the insertion of the anchor screw into the orifice of the bone implant, the first blocking member and the second blocking member are thus successively deformed between the open configuration and the closed configurations: the passage between these different configurations allows therefore authorizing this insertion of the anchor screw and automatically locking its position in the orifice.

It is important to note that this blocking of the anchor screw does not require any specific action from the practitioner other than screwing the anchor screw into the orifice: extracting the anchor screw from the orifice is automatically produced by the elastic deformation of the first blocking member and of the second blocking member caused by their interaction with the unidirectional detents of the anchor screw.

The invention also concerns a surgical kit including:
a bone implant as previously described, and
at least one anchor screw as previously described;

in which said anchor screw is adapted to cooperate with the anchor assembly of said bone implant such that:
the lower abutment of said anchor screw can be bearing with polyaxiality on the lower portion of the orifice of the anchor assembly of said bone implant so that, when said anchor screw is received in said orifice, the screw axis can form with the steering axis a screw angle with an adjustable measurement, and
the first blocking member and the second blocking member can come into contact with the unidirectional detents of said anchor screw when said anchor screw is received in said orifice, said first blocking member and second blocking member authorizing an insertion of said anchor screw into said orifice when they are in the open configuration and prevent an extraction of said anchor screw from said orifice when they are in a closed configuration, regardless of the measurement of the screw angle.

In an embodiment, the lateral ramps of the anchor screw are suitable to cooperate with the first blocking member and second blocking member, so as to apply, during an insertion movement of said anchor screw in the orifice of the bone implant, a constraint on said first blocking member and said second blocking member, said constraint allowing said first blocking member and second blocking member to be deformed so as to change them from a closed configuration to the open configuration and thus authorize said insertion movement of said anchor screw into said orifice.

According to a possibility, the bone implant and the anchor screws are such that the radial projections of the first blocking member and the radial projections of the second blocking member are respectively in contact with one of the blocking plates and another of the distinct blocking plates, when said anchor screw is received in the orifice of said bone implant and extends along the steering axis of said orifice.

Each blocking member and each blocking plate then extends orthogonally to the steering axis of the orifice and the blocking members have a large contact surface with the blocking plates, allowing effective blocking of the anchor screw in the orifice.

According to another possibility, at least one of the radial projections of the first blocking member and at least one of the radial projections of the second blocking member are each in contact with at least one respective blocking plate, when said anchor screw is received in the orifice of the bone implant and the screw angle is of non-zero measurement.

Each blocking member is thus in contact with at least one blocking plate when the anchor screw extends in an oblique direction relative to the steering axis.

According to yet another possibility, the radial projections of the first blocking member are in contact with at least two distinct blocking plates, and the radial projections of the second blocking member are in contact with at least two distinct blocking plates, when said screw anchor is received in the orifice of the bone implant and the screw angle is of non-zero measurement.

Each blocking member thus straddles at least one lateral ramp and is in contact with two distinct unidirectional detents.

According to yet another possibility, each of the radial projections of the first blocking member and each of the radial projections of the second blocking member is in contact with a blocking plate or a lateral ramp of the anchor screw, when said anchor screw is received in the orifice of the bone implant, regardless of the measurement of the screw angle.

In other words, each of the radial projections of the blocking members is permanently in contact with one or more unidirectional detents of the anchor screw, whatever the measurement of the screw angle: this anchor screw is thus reliably locked in the orifice, due to the large number of points of contact between the latter and the blocking members of the bone implant.

Other characteristics and advantages of the present invention will become apparent on reading the detailed description below of a non-limiting example of implementation, made with reference to the appended figures in which:

FIG. 1 is a perspective view of a bone implant according to the invention,

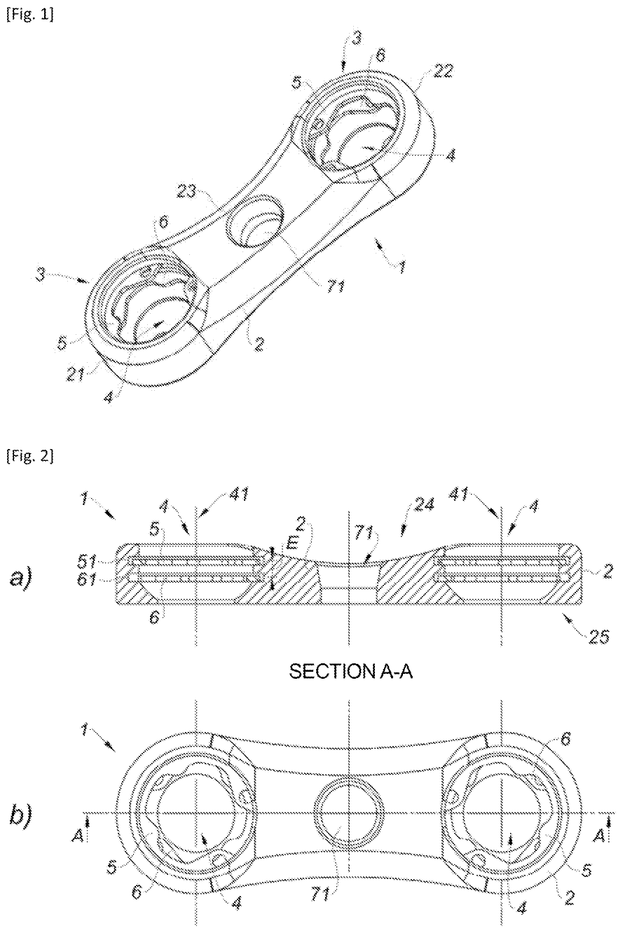

FIG. 2 is a front (FIG. 2a) and side (FIG. 2b) sectional view of a bone implant according to the invention,

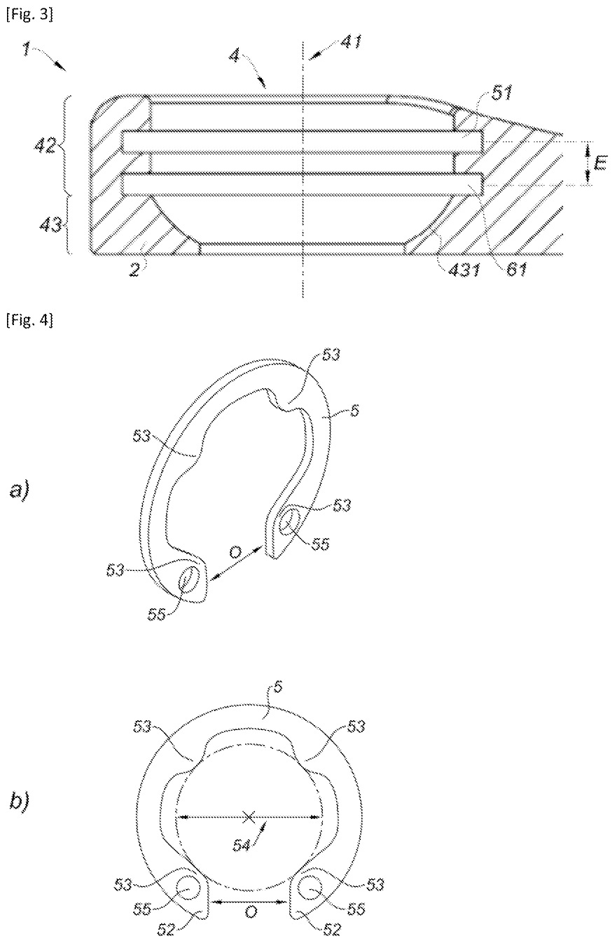

FIG. 3 is a detailed view of an orifice of a bone implant according to the invention,

FIG. 4 is a perspective (FIG. 4a) and front (FIG. 4b) view of a blocking member of a bone implant according to the invention,

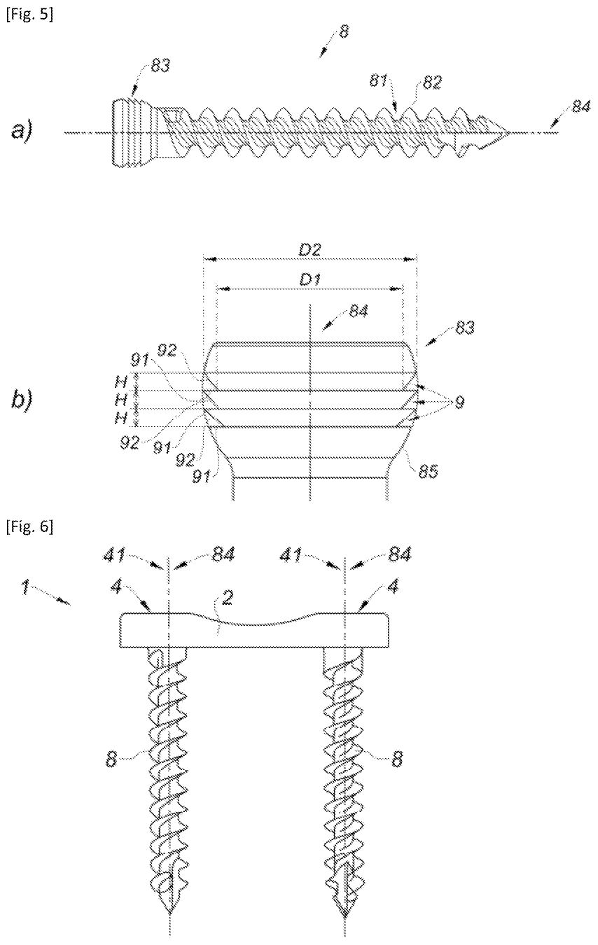

FIG. 5 is a perspective (FIG. 5a) and detailed (FIG. 5b) view of an anchor screw according to the invention,

FIG. 6 is a perspective view of a first case of use of a bone implant according to the invention and an anchor screw according to the invention,

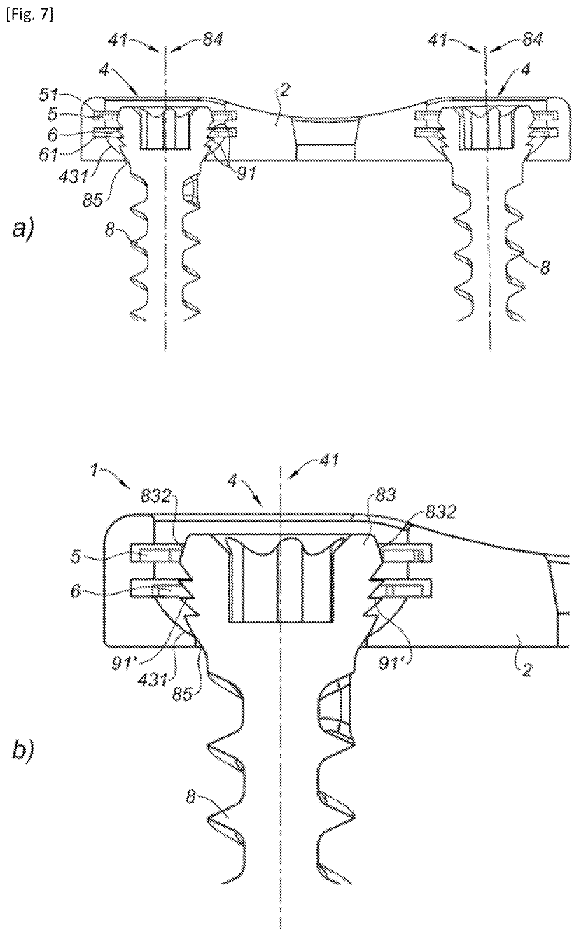

FIG. 7 is a sectional (FIG. 7a) and detailed (FIG. 7b) view of a bone implant according to the invention and an anchor screw according to the invention in the first case of use

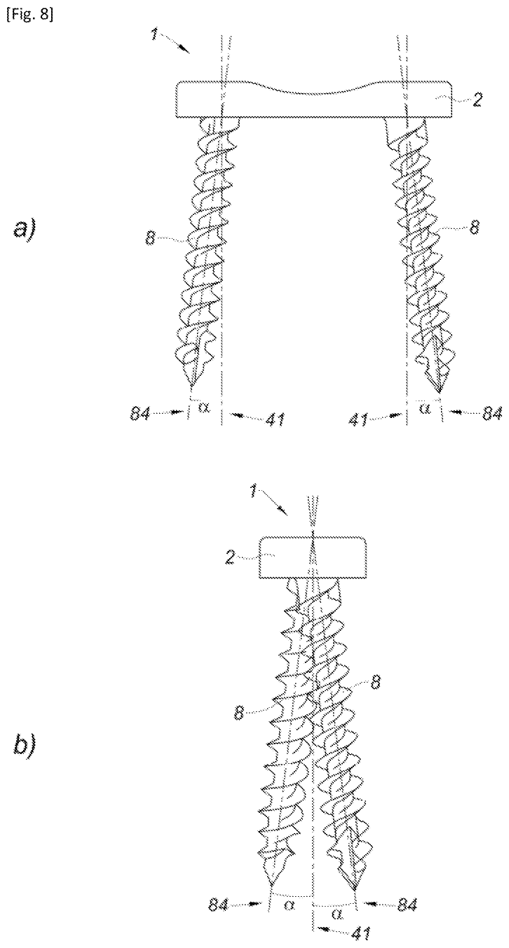

FIG. 8 is a perspective view in front (FIG. 8a) and side (FIG. 8b) view of a second case of use of a bone implant according to the invention and an anchor screw according to the invention,

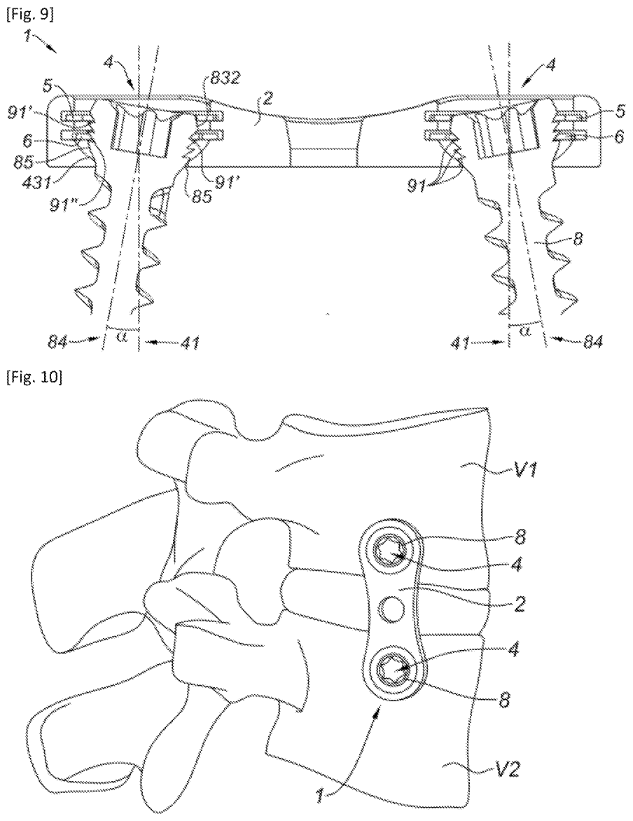

FIG. 9 is a sectional view of a bone implant according to the invention and an anchor screw according to the invention in the second case of use,

FIG. 10 is a perspective view of a bone implant according to the invention fixed to two vertebrae using anchor screws according to the invention,

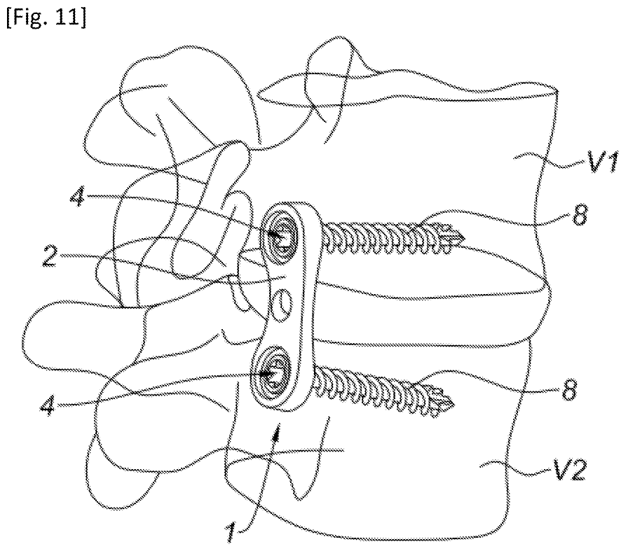

FIG. 11 is a transparent view of a bone implant according to the invention fixed to two vertebrae using anchor screws according to the invention.

FIG. 1 is a perspective view of a bone implant 1 according to the invention.

This bone implant 1 is in the form of a plate 2 provided with two anchor assemblies 3 positioned at two ends 21 and 22 of said plate 2, each of these anchor assemblies 3 including an orifice 4, a first blocking member 5 and a second blocking member 6.

These anchor assemblies 3 are intended to receive each an anchor screw making it possible to fix the bone implant 1 according to the invention on one or more bone structures, as will be described below, and for example on vertebrae.

The plate 2 also includes a hooking means 7, positioned at a central portion 23 of the plate 2, between the two anchor assemblies 3, and allowing the bone implant 1 to be fixed to a tool (not represented) adapted to facilitate handling thereof by a practitioner.

This attachment means includes an orifice 71, intended to cooperate with a projecting rod of said tool.

The structure and the geometry of the bone implant 1, and particularly the anchor assemblies 3, are best seen in FIGS. 2 and 3.

As visible in these FIGS. 2 and 3, the orifices 4 are formed through between an upper face 24 and a lower face 25 of the plate 2, and extend along a steering axis 41.

Each orifice 4 has an upper portion 42, opening towards the upper face 24, and a lower portion 43, opening towards the lower face 25, with very different geometries and functions:
the upper portion 42 fulfills the function of blocking an anchor screw received in the orifice 4 and includes a first peripheral housing 51 in which is housed the first blocking member 5, and a second peripheral housing 61 in which is housed the second blocking member 6, each of the first blocking member 5 and the second blocking member 6 being intended to be brought into contact with said anchor screw, as will be further detailed below, so as to prevent its extraction from the orifice 4;
the lower portion 43 has the function of allowing a bearing with polyaxiality of said anchor screw in the orifice 4 and has a bearing surface 431 of a generally spherical shape.

The first peripheral housing 51 and the second peripheral housing 61 are offset with respect to each other along the steering axis 41, and are mutually parallel and orthogonal to the steering axis 41. The first peripheral housing 51 and the second peripheral housing 61 are each in the form of an annular groove of generally circular shape, and opening only into the upper portion 42.

Thus, the first blocking member 5 and the second blocking member 6 are also positioned orthogonally to the steering axis 41 and distant from each other by a spacing distance E.

The first blocking member 5 is, in the single represented embodiment but in a non-limiting manner, of the circlip type: it is thus in the form of a split ring of circular shape, having two ends 52 distant from an opening distance O.

This first blocking member 5 is also elastically deformable and the opening distance O is adjustable: the first blocking member 5 can thus be deformed between an open configuration, in which the opening distance O is maximum, and closed configurations, in which the opening distance O can take smaller values.

This first blocking member 5 occurs naturally, when no external constraint is applied to it, in a rest configuration, in which the opening distance O is minimal. It should be noted that this opening distance O can be even smaller, when the first blocking member 5 is mounted inside its first peripheral housing 51.

The first blocking member 5 also includes four radial projections 53 defining an internal diameter 54: the passage of the first blocking member 5 between the closed or open rest configurations therefore results in a reduction (passage from the open configuration to a closed configuration) or an increase (from a closed configuration to the open configuration) of this inner diameter 54.

These radial projections 53 are here regularly distributed around the circumference of the first blocking member 5 so as to allow, as will be described below, to distribute the points of contact between the first blocking member 5 and an anchor screw received in the orifice 4, in order to achieve better blocking thereof in this orifice 4.

It should be noted that the first blocking member 5 also has orifices 55 positioned at the ends 52, making it possible, using a suitable tool (called «circlip pliers», not represented), to force the opening or the closing of the first blocking member 5 and to position the latter in the first peripheral housing 51.

The second blocking member 6 may be identical to the first blocking member 5 represented in FIG. 4, or have a different structure, this second blocking member 6 however having to be able to be elastically deformable between an open configuration and closed configurations.

The first blocking member 5 and the second blocking member 6 are designed to cooperate with a suitable anchor screw 8, represented in FIG. 5, when the latter is received in the orifice 4.

This anchor screw 8 includes a rod 81 extending along a screw axis 84, having a thread 82 and intended to be introduced into a bone structure, and a head 83, intended to be introduced into the orifice 4 and to cooperate with the first blocking member 5 and the second blocking member 6.

The head 83 has the general shape of a spherical portion.

Particularly, the head 83 has a lower abutment 85 and includes on its circumference three unidirectional detents 9 successively disposed along the screw axis 84, each of these unidirectional detents 9 being formed by:
a blocking plate 91, which is flat and orthogonal to the screw axis 84, and
a lateral ramp 92, joining two successive blocking plates 91 and extending obliquely with respect to the screw axis 84.

Each unidirectional detent 9 thus appears as a notch or an indentation of height H formed at the periphery of the head 83 of the anchor screw 8 and corresponds (along the screw axis 84 and in a direction going from the rod 81 towards the head 83) to an increase in the diameter of the anchor screw 8 between a minimum diameter D1 and a minimum diameter D2.

It will be noted that, in the represented embodiment, each unidirectional detent 9 has the same height H and the lateral ramps 92 are mutually parallel, but other possibilities are also possible.

It will also be noted that each unidirectional detent 9 has a symmetry of revolution around the screw axis 84.

This structure of the unidirectional detents 9 of the anchor screw 8 is particularly suitable to allow automatic blocking of the latter in the orifice 4, thanks to its cooperation with the first blocking member 5 and the second blocking member 6 disposed around the head 83 in position, as seen in FIGS. 7 and 9 below.

Indeed, the unidirectional detents 9 are dimensioned so that:
the diameter D2 corresponds to the internal diameter 54 of the first blocking member 5 and of the second blocking member 6 when the latter are in the open configuration, and
the diameter D1 corresponds to the internal diameter 54 of the first blocking member 5 and of the second blocking member 6 when the latter are in a closed configuration.

In this way, the diameter of the head 83 of the anchor screw 8 always remaining greater than the internal diameter 54 of the first blocking member 5 and of the second blocking member 6 when the latter are in the rest configuration, the first blocking member 5 and second blocking member 6 are permanently in contact with this head 83 during the insertion of the anchor screw 8 into the orifice 4, and, more particularly, the lateral projections 53 of the first blocking member 5 and the second blocking member 6 are in uninterrupted contact with a blocking plate 91 and/or a lateral ramp 92.

It is thus possible to authorize an insertion of the anchor screw 8 in the orifice 4 and to ensure effective automatic blocking of this anchor screw 8 in the orifice 4: during this insertion movement, the translation of the lateral ramps along the steering axis 41 of the orifice has the effect of exerting a radial constraint on the first blocking member 5 and the second blocking member 6 and thus gradually increasing the internal diameter 54 of the first blocking member 5 and of the second blocking member 6, from the value of the diameter D1 to that of the diameter D2.

Once the diameter D2 is reached, the internal diameter 54 of the first blocking member 5 and of the second blocking member 6 suddenly changes to the diameter D1, before gradually increasing again to the diameter D2, etc.

Due to their contact with the lateral ramps 92, the first blocking member 5 and the second blocking member 6 therefore undergo, during the insertion of the anchor screw 8 into the orifice 4, successive deformations, from a closed configuration to the open configuration, before the anchor screw 8 reaches a blocking position illustrated by the following FIGS. 6 to 9.

In this blocking position, the lower abutment 85 of the anchor screw 8 is in contact with the surface 431 of the lower portion of the orifice 4 and the head 83 is entirely contained in the orifice 4, the first blocking member 5 and the second blocking member 6 preventing, by their cooperation with the unidirectional detents 9, any extraction movement of the anchor screw 8 out of the orifice 4.

FIGS. 6 and 7 represent a first case of use of the bone implant 1 according to the invention, in which each anchor screw 8 is inserted into an orifice 4 of the bone implant 1 along the steering axis 41 of said orifices 4.

Particularly, the steering axis 41 then coincides with the screw axis 84.

In this first case of use, as can be seen in FIGS. 7a and 7b, the blocking plates 91 and the first blocking member 5 and the second blocking member 6 are orthogonal to the steering axis 84 and parallel to each other: it is thus possible to achieve a wide surface contact between at least one blocking plate 91 and one of the first blocking member 5 and of the second blocking member 6.

For example, in this FIG. 7, the identified blocking plate 91′ is in surface contact, orthogonally to the steering axis 41, with the second blocking member 6 over the entire circumference of the head 83 of the anchor screw 8.

Because of this contact, it is then impossible to extract the anchor screw 8 from the orifice 4 because the blocking plate 91′ would then constitute a retainer against which the second blocking member 6 would come into abutment, preventing such an extraction movement.

It will be noted, moreover, that the first blocking member 5 is not here in contact with a unidirectional detent 9 but with an upper end 832 of the head 83 of the anchor screw 8: this first blocking member 5 participating all the same to the blocking of the anchor screw 8 in the orifice 4 by the pressure force which it exerts radially on the latter (the diameter of the upper end being greater than the diameter D1).

It may be advantageous to size the head 83 of the anchor screw 8 so that the height H of the unidirectional detents 9 is equal to the spacing distance E between the first blocking member 5 and the second blocking member 6: this first blocking member 5 and this second blocking member 6 will then be each in surface contact with a distinct blocking plate 91 and the blocking of the anchor screw 8 in the orifice 4 will be improved thereby.

Thus, thanks to the cooperation between the unidirectional detents 9 and the first blocking member 5 and/or the second blocking member 6, it is possible to automatically block the anchor screw 8 in the orifice 4 when this anchor screw 8 extends along the steering axis 41.

FIGS. 8 and 9 represent a second case of use of the bone implant 1 according to the invention, in which each anchor screw 8 is inserted into an orifice 4 of the bone implant 1 in an oblique direction with respect to the steering axis 41 of said orifices 4.

The screw axis 84 of each anchor screw 8 then forms a screw angle α with the steering axis 41 of the orifice 4 in which they are inserted.

In FIGS. 8a and 8b, this screw angle is represented in a front view (FIG. 8a) and a side view (FIG. 8b) and has the same measurement for each of the anchor screws 8.

It is however conceivable that each anchor screw 8 has a screw angle α of different measurement.

In this second case of use, as can be seen in FIG. 9, the blocking plates 91 are not orthogonal to the steering axis 41 of the orifice 4: an extended surface contact between a same blocking plate and the first blocking member 5 and/or the second blocking member 6 is therefore impossible, this first blocking member 5 and this second blocking member 6 being themselves orthogonal to the steering axis 41.

The blocking of the anchor screw 8 in the orifice 4 is here achieved by:
a contact between the second blocking member 6 and two distinct blocking plates, identified 91″ and 91′″; and
a contact between the first blocking member 5 and the blocking plate 91″ on the one hand and between the first blocking member 5 and the upper end 832 of the head 83 on the other hand.

Thus, the second blocking member 6 is in the position of overlapping several unidirectional detents 9 and is in contact with two distinct blocking plates: this contacting mode is made possible in particular by the presence of the radial projections 53 which allow the second blocking member 6 to «penetrate» more deeply into the unidirectional detents 9.

As previously, placing the second blocking member 6 in contact with the blocking plates 91″ and 91′″, and that of the first blocking member 5 with the blocking plate 91″ make it possible to effectively block the anchor screw 8 in the orifice 4, these blocking plates 91″ and 91′″ forming retainers against which the first blocking member 5 and the second blocking member 6 would abut during an extraction movement of the anchor screw 8 along the screw axis 84.

The contact between the first blocking member 5 and the upper end 832 of the head 83 also contributes to this blocking, by the pressure force which it exerts radially thereon.

Here again, it may be advantageous to size the head 83 of the anchor screw 8 so that the height H of the unidirectional detents 9 is equal to the spacing distance E separating the first blocking member 5 and the second blocking member 6: this first blocking member 5 and this second blocking member 6 will then be each in contact with two distinct blocking plates and the blocking of the anchor screw 8 in the orifice 4 will be improved thereby.

It is important to note that the insertion of the anchor screw 8 in the orifice 4 in an oblique position with respect to the steering axis 41 (as represented in FIGS. 8 and 9) is made possible by the particular geometry of the lower portion 82 of the orifice 4.

Indeed, this lower portion 43 has a bearing surface 431 having a much greater flaring than the flaring of the lower abutment 85 of the anchor screw 8.

Thus, once this bearing surface 431 and this lower abutment 85 have been brought into contact (annular contact), the lower portion 43 of the orifice 4 has a sufficient width to allow the inclination of the anchor screw 8 in this same orifice 4 (a portion of the head 83 of the anchor screw 8 then being received in the lower portion 43): it is therefore possible to adjust the measurement of the screw angle between a zero value (configuration represented by the preceding FIGS. 6 and 7, in which the screw axis 84 coincides with the steering axis 41) and a maximum value (configuration not represented in which the head 83 of the anchor screw 8 is in contact with the bearing surface 431).

In the represented embodiment, the screw angle can take any value included in the interval between the zero value and the maximum value: other embodiments can be envisaged in which the screw angle cannot take only a limited number of values in this same interval.

This possibility of adjusting the inclination of the anchor screw 8 in the orifice 4 is called «polyaxiality» and gives the practitioner great flexibility concerning the operation of fixing the bone implant 1 according to the invention on one or more bone structures, the latter being able to adapt the direction of screwing of the anchor screws 8 to each particular patient or to each mode of use of the bone implant 1.

The bone implant 1 according to the invention is therefore well provided with blocking means allowing automatic and reliable blocking of the anchor screw 8 in the orifices 4, regardless of the inclination of these anchor screws 8 with respect to the steering axis 41 of the orifices 4.

The following FIGS. 10 and 11 illustrate an example of use of the bone implant 1 according to the invention, in which the latter is a spinal implant or spinal arthrodesis implant providing a fusion of two vertebrae V1, V2, this bone implant 1 being fixed by means of two anchor screws 8 to two adjacent vertebrae V1 and V2, each anchor screw 8 being screwed into a distinct vertebra V1 or V2. The bone implant 1 then allows stabilization of the spine by a fusion of two vertebrae V1, V2.

Other uses and modes of using such a bone implant 1 according to the invention can be envisaged, it being possible for the latter to be fixed on one or more bone structures of different nature and geometry. It is also conceivable to adapt the shape of the plate 2 and the positioning of the orifices 4 to particular and specific implementations of the invention.

1. A bone implant, adapted to be fixed on at least one bone structure, including a plate provided with at least one anchor assembly provided with an orifice extending along a steering axis and adapted to receive an anchor screw extending along a screw axis, the anchor assembly comprising a first blocking member arranged in a first peripheral housing formed in the orifice,
the bone implant being characterized in that wherein:
the anchor assembly further comprises at least one second blocking member disposed in a second peripheral housing formed in the orifice, the second peripheral housing being offset relative to the first peripheral housing along the steering axis,
the orifice includes a lower portion offering, when the anchor screw is received in the orifice, a bearing with polyaxiality to the anchor screw so that the screw axis can form with the steering axis an screw angle with an adjustable measurement, and
the first blocking member and second blocking member are elastically deformable between an open configuration, in which they authorize an insertion of the anchor screw in the orifice, and at least a closed configuration, in which they prevent an extraction of the anchor screw from the orifice, whatever the measurement of the screw angle.

2. The bone implant according to claim 1, wherein the measurement of the screw angle is adjustable within a range comprised between 0 to 15 degrees.

3. The bone implant according to claim 1, wherein the first blocking member and the second blocking member are elastically deformable in the direction of a natural return of the open configuration towards a closed configuration in the absence of an external constraint which is applied to them.

4. The bone implant according to claim 1, wherein the first blocking member and the second blocking member are each of the split ring or circlip type.

5. The bone implant according to claim 4, wherein the first blocking member and the second blocking member each extend in a direction orthogonal to the steering axis of the orifice.

6. The bone implant according to claim 1, wherein each of the first blocking member and the second blocking member has at least two distinct radial projections, the radial projections being intended to be brought into contact with the anchor screw when the anchor screw is received in the orifice and when the first blocking member and the second blocking member are in a closed configuration.

7. The bone implant according to claim 6, wherein each of the first blocking member and the second blocking member has at least four distinct radial projections.

8. The bone implant according to claim 1, wherein the plate comprises at least two anchor assemblies.

9. An anchor screw adapted for a bone implant in accordance with claim 1, the anchor screw being adapted to cooperate with the anchor assembly of the bone implant, the anchor screw having a rod extending along a screw axis and a head provided with:
a lower abutment suitable to bear with polyaxiality on the lower portion of the orifice of the anchor assembly so that, when the anchor screw is received in the orifice, the screw axis can form with the steering axis a screw angle with the adjustable measurement; and
at least two unidirectional detents, the unidirectional detents being provided to cooperate with the first blocking member and the second blocking member when the anchor screw is received in the orifice of the anchor assembly, so that the first blocking member and the second blocking member authorize an insertion of the anchor screw in the orifice when they are in the open configuration and prevent extraction of the anchor screw from the orifice when they are in a closed configuration, regardless of the screw angle measurement.

10. The anchor screw according to claim 9, wherein the unidirectional detents have a symmetry of revolution centered on the screw axis.

11. The anchor screw according to claim 9, wherein the unidirectional detents are formed by:
blocking plates, disposed successively around the head of the anchor screw, mutually parallel and orthogonal to the screw axis, and
lateral ramps joining two successive blocking plates, the blocking plates allowing, by contact with the first blocking member and the second blocking member when the anchor screw is received in the orifice of the bone implant and when the first blocking member and the second blocking member are in a closed configuration, to prevent an extraction of the anchor screw out of the orifice of the bone implant.

12. The anchor screw according to claim 11, wherein the lateral ramps are configured to apply, during an insertion movement of the anchor screw in the orifice of the bone implant, a constraint on the first blocking member and the second blocking member, the constraint making it possible to deform the first blocking member and second blocking member so as to change them from a closed configuration to an open configuration.

13. A surgical kit including:
a bone implant in accordance with claim 1, and
at least one anchor screw adapted for a bone implant, the anchor screw being adapted to cooperate with the anchor assembly of the bone implant, the anchor screw having a rod extending along a screw axis and a head provided with:
a lower abutment suitable to bear with polyaxiality on the lower portion of the orifice of the anchor assembly so that, when the anchor screw is received in the orifice, the screw axis can form with the steering axis a screw angle with the adjustable measurement; and
at least two unidirectional detents, the unidirectional detents being provided to cooperate with the first blocking member and the second blocking member when the anchor screw is received in the orifice of the anchor assembly so that the first blocking member and the second blocking member authorize an insertion of the anchor screw in the orifice when they are in the open configuration and prevent extraction of the anchor screw from the orifice when they are in a closed configuration, regardless of the screw angle measurement;
wherein the anchor screw is adapted to cooperate with the anchor assembly of the bone implant such that:
the lower abutment of the anchor screw can be bearing with polyaxiality on the lower portion of the orifice of the anchor assembly of the bone implant so that, when the anchor screw is received in the orifice, the screw axis can form with the steering axis a screw angle with the adjustable measurement, and
the first blocking member and the second blocking member can come into contact with the unidirectional detents of the anchor screw when the anchor screw is received in the orifice, the first blocking member and second blocking member authorizing an insertion of the anchor screw in the orifice when they are in the open configuration and prevent extracting the anchor screw from the orifice when they are in a closed configuration, regardless of the measurement of the screw angle.

14. The surgical kit according to claim 13, wherein the bone implant wherein the first blocking member and the second blocking member are elastically deformable in the direction of a natural return of the open configuration towards a closed configuration in the absence of an external constraint which is applied to them and the anchor screw wherein the lateral ramps are configured to apply, during an insertion movement of the anchor screw in the orifice of the bone implant, a constraint on the first blocking member and the second blocking member, the constraint making it possible to deform the first blocking member and second blocking member so as to change them from a closed configuration to an open configuration, the lateral ramps of the anchor screw being suitable to cooperate with the first blocking member and second blocking member, so as to apply, during an insertion movement of the anchor screw in the orifice of the bone implant, a constraint on the first blocking member and the second blocking member the constraint making it possible to deform the first blocking member and second blocking member so as to make them change from a closed configuration to the open configuration and thus authorize the insertion movement of the anchor screw into the orifice.

15. The surgical kit according to claim 13, wherein the bone implant wherein the first blocking member and the second blocking member each extend in a direction orthogonal to the steering axis of the orifice and the anchor screw wherein the unidirectional detents are formed by:
blocking plates, disposed successively around the head of the anchor screw, mutually parallel and orthogonal to the screw axis, and
lateral ramps joining two successive blocking plates,
the blocking plates allowing, by contact with the first blocking member and the second blocking member when the anchor screw is received in the orifice of the bone implant and when the first blocking member and the second blocking member are in a closed configuration, to prevent an extraction of the anchor screw out of the orifice of the bone implant, so that the radial projections of the first blocking member and the radial projections of the second blocking member are respectively in contact with one of the blocking plates and another of the distinct blocking plates, when the anchor screw is received in the orifice of the bone implant and extends along the steering axis of the orifice.

16. The surgical kit according to claim 15, wherein at least one of the radial projections of the first blocking member and at least one of the radial projections of the second blocking member are each in contact with at least one respective blocking plate, when the anchor screw is received in the orifice of the bone implant and the screw angle is of a non-zero measurement.

17. The surgical kit according to claim 16, wherein the radial projections of the first blocking member are in contact with at least two distinct blocking plates, and the radial projections of the second blocking member are also in contact with at least two distinct blocking plates, when the anchor screw is received in the orifice of the bone implant and the screw angle is of a non-zero measurement.

18. The surgical kit according to claim 15, wherein each of the radial projections of the first blocking member and each of the radial projections of the second blocking member is in contact with a blocking plate or a lateral ramp of the anchor screw, when the anchor screw is received in the orifice of the bone implant, regardless of the measurement of the screw angle.
